(19) United States
(12) Patent Application Publication
(10) Pub. No.: US 2022/0105276 A1
(43) Pub. Date: Apr. 7, 2022

(54) METHODS AND SYSTEMS FOR TRACKING DOSAGE INFORMATION OF ELECTRONICALLY ENABLED INJECTION DEVICES

(71) Applicant: Sanofi, Paris (FR)

(72) Inventors: Timothy Lynch, Reading, MA (US); Sean Phillips, Lancaster, MA (US); Gregory Stern, Bridgewater, NJ (US); Kelsey Yevak, Charlestown, MA (US)

(21) Appl. No.: 17/449,591

(22) Filed: Sep. 30, 2021

Related U.S. Application Data
(60) Provisional application No. 63/086,931, filed on Oct. 2, 2020.

(30) Foreign Application Priority Data
Nov. 17, 2020 (EP) 20315452.1

(51) Int. Cl. A61M 5/315; A61M 5/20; A61M 5/31; G16H 20/17

(52) U.S. Cl. CPC A61M 5/31541; A61M 5/31546; G16H 20/17; A61M 5/3146; A61M 5/20

(57) ABSTRACT

Dosing information of an electronically enabled injection device is tracked. Dosing information indicating an amount of medicament dialed or dispensed by the electronically enabled injection device is received. A dose type of a plurality of potential dose types is selected. A user interface indicating the selected dose type is displayed.

### CROSS-REFERENCE TO RELATED APPLICATIONS

This application claims priority to U.S. Provisional Patent Application No. 63/086,931, filed on Oct. 2, 2020, and to European Patent Application No. 20315452.1, filed on Nov. 17, 2020, the entire contents of which are incorporated herein by reference.

### TECHNICAL FIELD

This disclosure is directed to methods and systems for tracking dosage information of electronically enabled injection devices.

### BACKGROUND

Electronically enabled injection devices assist users in safely administering a medicament and can also enable transmission of treatment data to medical staff. Electronically enabled injection devices can include an electronic component configured to provide continuous active sensing and connectivity properties. For example, using electronic sensors, an injection device can capture and transmit data related to an amount of medicament (dose of medicament) that has been dialed or expelled from the injection device.

Some injection devices, such as some insulin injection pens, are primed before an injection to remove air, which may collect during use, from a needle and/or cartridge of the injection device. Such a priming step can help to ensure that the injection device functions properly. For instance, users of an injection device may perform a priming operation before injecting themselves with medicament. The priming operation typically includes dialing a priming dose and pressing an injection button while holding the injection device with the needle pointed upwards. The priming dose may be, for example, two units of medicament, while the injection dose may be twenty units of medicament or more.

### SUMMARY

Implementations of the present disclosure describe systems and methods (technology) for distinguishing between a safety/priming dose and an injection dose for tracking of injection/dosing information. In some implementations, a computing device (e.g., a laptop, desktop, tablet, smartphone, etc.) receives dosing data from an electronically enabled injection device (e.g. an autoinjector). The dosing data indicates a dosing amount that was either dialed or dispensed using the injection device. The computing device can be used to track the dosing amounts over time. In some implementations, the computing device is configured to distinguish between priming doses and injection doses. Priming doses can be ignored or tracked/labeled separately from injection doses.

In an aspect, a computer-implemented method tracks dosing information of an electronically enabled injection device. The method includes receiving, by a computing device, dosing information indicating an amount of medicament dialed or dispensed by the electronically enabled injection device. The method includes automatically selecting, by the computing device and based on the amount of medicament, a dose type of a plurality of potential dose types. The method includes rendering, by the computing device and on a display of the computing device, a user interface indicating the selected dose type.

In an aspect, a system tracks dosing information of an electronically enabled injection device. The system one or more processors. The system includes computer memory storing instructions that, when executed by the processors, cause the processors to perform operations. The operations include receiving, by a computing device, dosing information indicating an amount of medicament dialed or dispensed by the electronically enabled injection device. The operations include automatically selecting, by the computing device and based on the amount of medicament, a dose type of a plurality of potential dose types. The operations include rendering, by the computing device and on a display of the computing device, a user interface indicating the selected dose type.

In an aspect, a computer-readable medium is used for tracking dosing information of an electronically enabled injection device. The medium stores instructions that, when executed by one or more processors, cause the processors to perform operations. The operations include receiving, by a computing device, dosing information indicating an amount of medicament dialed or dispensed by the electronically enabled injection device. The operations include automatically selecting, by the computing device and based on the amount of medicament, a dose type of a plurality of potential dose types. The operations include rendering, by the computing device and on a display of the computing device, a user interface indicating the selected dose type.

In an aspect, the plurality of dose types includes a priming dose and an injection dose.

In an aspect, the user interface comprises one or more user selectable icons that allows a user to confirm the selected dose type.

In an aspect, the user interface comprises one or more user selectable icons that allow a user to adjust the dose type from the selected dose type.

In an aspect, the plurality of dose types comprises a priming dose and an injection dose; and selecting a dose type comprises determining if the amount of medicament exceeds a threshold amount

Systems in accordance with the present disclosure can include any combination of the aspects and features described herein. That is to say that systems in accordance with the present disclosure are not limited to the combinations of aspects and features specifically described herein, but also include any combination of the aspects and features provided.

The details of one or more embodiments of the present disclosure are set forth in the accompanying drawings and the description below. Other features and advantages of the present disclosure will be apparent from the description and drawings, and from the claims.

Implementations of the present disclosure can provide one or more of the following advantages. Unlike conventional dose tracking technology, the technology described in this specification can distinguish between priming doses and injection doses, which can improve the dose tracking technologies ability to accurately track injection dosage information over time. Unlike conventional dose tracking technology, a user can use the technology described in this specification to confirm that a dose has been tracked accurately. Unlike conventional dose tracking technology, user input requirements can be reduced.

### DESCRIPTION OF DRAWINGS

FIG. 1 is an exploded view of a medicament injection system that includes an injection device and an external device, according to one or more implementations of the present disclosure.

FIG. 2 schematically illustrates a stopper having an electronics assembly, which can be used in an injection device according to one or more implementations of the present disclosure.

FIGS. 3A-3B illustrate user interfaces for tracking dosage information, according to one or more implementations of the present disclosure.

FIG. 4 illustrates a method for tracking dosage information, according to one or more implementations of the present disclosure.

FIG. 5 is a schematic illustration of example computer systems that can be used to execute implementations of the present disclosure.

Like reference symbols in the various drawings indicate like elements.

### DETAILED DESCRIPTION

An electronically enabled injection device can be configured to communicate dosage information to a computing device. For example, an injection device can include sensory and other electronics to capture dosing information indicating an amount (dose) of medicament that was dialed and/or dispensed by the injection device. The injection device can include an interface, such as a transceiver, to wirelessly transmit the dosing information to a computing device. The computing device can receive and process the dosing information to allow a user to track the dosing information over time. Tracking dosing information over time can enable a user to determine how much medicament the user has injected into his or her body, the date/time of each injection, and whether a prescribed injection was missed or accurately completed by the user. However, it may be difficult to accurately track such injection information when the tracking device does not distinguish between injection doses and priming doses. For example, without such a mechanism to distinguish between the types of doses, a priming dose followed by an injection dose may be tracked as two injections and/or tracked as a higher injection dose than was actually delivered into the patient. Accordingly, it can be advantageous to configure the dose tracking device (e.g., the computing device) to distinguish a priming dose from an injection dose.

Implementations of the present disclosure provide mechanisms that can advantageously distinguish between a priming dose and an injection dose. In some implementations, a computing device used to track dosing information is configured to determine whether received dosing information indicates a priming dose or an injection dose. In some implementations, if the dosing information indicates a dose that does not exceed a threshold dose, it is determined that the dose is a priming dose. In some implementations, the computing device is caused to render a user interface that allows a user to confirm whether a dose was a priming dose or an injection dose. In some implementations, the user interface indicates that a dose was determined to be one of a priming dose or an injection dose, and the user can use one or more selectable icons of the user interface to correct the determination by indicating that the dose was the other of a priming dose or injection dose. Priming doses can be ignored or tracked/labeled separately from injection doses. Accordingly, implementations of the described technology can ensure dosing information is being more accurately tracked, while reducing manual user input requirements for the user, which can be particularly advantageous for users with mental or reading impairments.

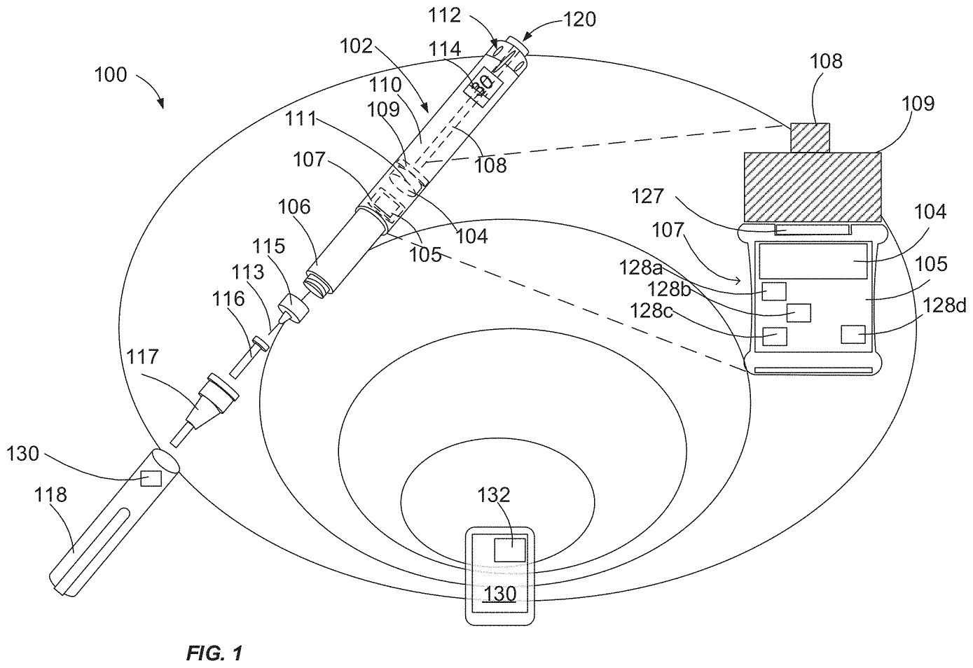

FIG. 1 illustrates an exploded view of a medicament injection system 100, according to one or more implementations of the present disclosure. The medicament injection system 100 can be configured to assist a user in injecting a fluid (e.g., a medicament) and facilitate sharing of medical data. The shown medicament injection system 100 includes an injection device 102 and an external device 130. The injection device 102 is an electronically enabled injection device. The injection device 102 can, for example, be a pre-filled, disposable injection pen or the injection device 102 can be a reusable injection pen with replaceable medicament reservoirs 106. In some implementations, the injection device 102 is capable of communicating with the external device 130. In some implementations, the injection device 102 is capable of transmitting to the external device 130 operational data (e.g., data related to time of start of usage of injection device 102 and temperature of injection device 102 during use and storage) and corresponding treatment data (e.g., amount of medicament dispensed and/or dialed, elapsed time for medicament to be dispensed by the injection device 102, and so forth). In some implementations, the injection device 102 is associated with an identifier that is used by the external device 130 to uniquely identify the injection device 102.

The injection device 102 includes a housing 110 and a needle assembly 115. The housing 110 includes an energy source 104, an electronics assembly 105, a medicament reservoir 106, a stopper 107, a plunger rod 108, a plunger head 109, a priming component (e.g., dosage knob) 112, a dosage window 114, and an injection button 120. The housing 110 can be molded from a medical grade plastic material, such as a thermoplastic polymer or liquid crystal polymer. In some implementations, the housing 110 includes two parts—a body part which houses dosing mechanics elements (e.g., the dosage knob) and a cartridge holder part which houses the medicament reservoir 106 and provides a needle hub. In some implementations, the stopper 107 includes at least one of the electronics assembly 105 or the energy source 104.

The medicament reservoir 106 is configured to contain a fluid medicament. The medicament reservoir 106 can be a conventional, generally cylindrical, disposable container, such as a cartridge or a syringe, used to package prepared fluids, such as medicaments, anesthetics and the like. In some implementations, one end of the medicament reservoir 106 has a pierceable membrane, which receives an inward end of needle 113 in sealing engagement. A dose of the contained medicament can be set by turning the dosage knob 112. The dosage knob 112 or a sleeve attached thereto can be provided with values representing different dosages printed or otherwise visible on its outer surface. The selected dose is displayed via dosage window 114, for instance in multiples of so-called International Units (IU), where one IU can be the biological equivalent of about 45.5 micrograms of pure crystalline medicament (e.g., 1/22 mg). An example of a selected dose displayed in dosage window 114 may for instance be 30 IUs, In some implementations, the selected dose is displayed differently, for instance by an electronic display (e.g., the dosage window 114 may take the form of an electronic display). Turning the dosage knob 112 can cause a mechanical click sound to provide acoustical feedback to a user. The numbers displayed in dosage window 114 can be printed on a sleeve that is contained in housing 110 and mechanically interacts with a plunger head 109 that is fixed at the end of the plunger rod 108 and pushes the stopper 107 of the medicament reservoir 106.

The plunger head 109 (e.g., a front end of the plunger rod 108) is operable to expel a portion of the fluid by displacing the stopper 107 within the medicament reservoir 106, such that a position of the stopper 107 is associated with an amount of the fluid within the injection device 102. In some implementations, the plunger rod 108 is mounted to the plunger head 109. In other implementations, the plunger rod 108 and the plunger head 109 are separate components. In some implementations, the plunger head 109 is mounted to the stopper 107.

The stopper 107 is a flexible stopper, such as a rubber stopper. The stopper 107 can include a rigid member surrounding the stopper 107. In some implementations, the stopper 107 is a rigid stopper with a sealing component. The stopper 107 can have an outwardly projecting rim matching the geometry and dimensions of the energy source 104. The stopper 107 can be of a sufficient length so that the stopper 107 is not ripped or twisted when being engaged by the plunger head 109. In some implementations, the stopper 107 includes the energy source 104 and the electronics assembly 105, among other components, as described in more detail below. In such implementations, the stopper 107 includes sufficient volume to house these components.

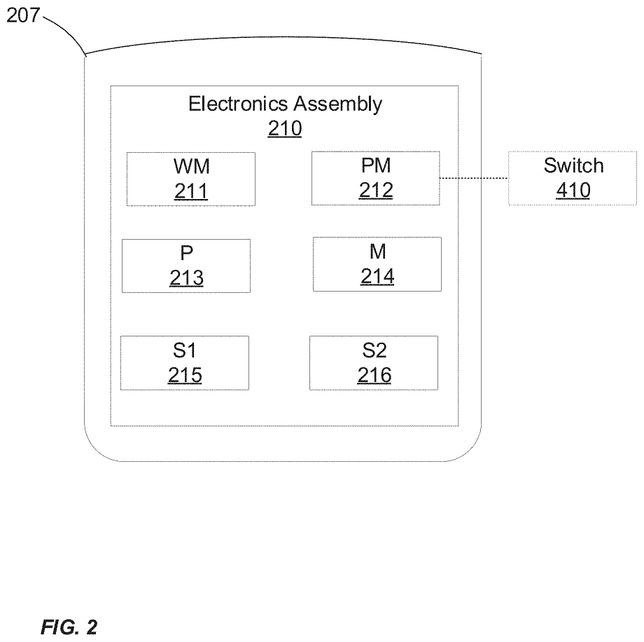

The electronics assembly 105 includes one or more sensors 128b. In some implementations, the one or more sensors 128b include at least one of a force sensor, a pressure sensor, or a position sensor. In some implementations, the energy source 104 provides minimal power for allowing the one or more sensors 128b enough power to operate sufficiently. In some implementations, the one or more sensors 128b have a separate power source, or have their own power source. As will be discussed later in more detail with reference to FIG. 2, in some implementations, the electronics assembly 105 is capable of detecting an amount of medicament expelled from or dialed by the injection device 102 based on signals generated by the one or more sensors 128b. The electronics assembly 105 also includes an electromechanical switch 127. The electromechanical switch 127 is an electrical switch, such as a piezo switch, that is capable of generating an electrical charge based on a force being applied to the switch 127 (i.e., upon activation of the switch 127). In some implementations, the electromechanical switch 127 is located on an inner surface of the stopper 107. In other implementations, the electromechanical switch 127 is located on an external surface of the stopper 107.

The energy source 104 can be a disposable or rechargeable battery, such as a 1.5V-5 V silver-oxide or lithium battery (e.g., SR626, SR516, SR416) or a super capacitor. In some implementations, the energy source 104 includes a plurality of batteries (e.g., two 1.5V batteries). The energy source 104 is communicatively coupled to the electromechanical switch 127. The electronics assembly 105 includes one or more electronic components configured to perform and/or assist with one or more functions of the injection device 102 (e.g., the ejection of the medicament) upon activation of the energy source 104. For example, the electronics assembly 105 can include one or more processors 128a, one or more sensors 128b, an antenna 128c, and a motor 128d. In some implementations, the motor 128d is configured to advance in micro-step increments to dispense a particular amount of medicament. In some implementations, the one or more sensors 128b are configured to provide, to the one or more processors 128a, a signal (e.g., a voltage), which is proportional to the amount of medicament dispensed or amount of medicament remaining in the medicament reservoir 106.

The injection device 102 can be used for several injection processes until either the medicament reservoir 106 is empty or the expiration date of the injection device 102 (e.g., 28 days after the first use) is reached. Before using the injection device 102 for the first time, it may be necessary to perform a priming operation to couple the energy source 104 to the electric component and/or to remove air from medicament reservoir 106 and needle 113. For instance, the priming operation can include selecting (or dialing) two units of medicament and pressing the injection button 120 while holding injection device 102 with the needle 113 upwards.

In some implementations, the one or more processors 128a include a microprocessor. In some implementations, the microprocessor is a microcontroller, for example, a combination of microprocessor components and other components formed in a single package. The microprocessor can be an arithmetic and/or a logic unit array. The one or more processors 128a are capable of processing one or more signals received from the other electronic components of the electronics assembly 105 (e.g. the sensors 128b) and transmitting a signal to the antenna 128c. For example, the one or more processors 128a can be configured to execute operations on received data to generate output data. In some implementations, the one or more processors 128a are capable of determining the amount of the fluid within the injection device 102 based at least in part on an electrical signal, and transmitting the data including information related to the amount of the fluid to the antenna 128c, which can then transmit it to the external device 130.

The antenna 128c can be a Bluetooth or near-field communication (NFC) antenna. The antenna 128c is capable of transmitting signals to the one or more processors 128a and to the external device 130. For example, the signals transmitted by the antenna 128c can include the amount of the fluid in the medicament reservoir 106, values measured by each of the one or more sensors 128b, and the identifier of the injection device 102. A communication field 134 can be a Bluetooth field or an NFC field, generated by the external device 130. The external device 130 can be a computing device, such as a smartphone, tablet, desktop computer, laptop computer, and so forth. The external device 130 can include a Bluetooth or a RF module, a transmitter, a receiver, and an external processor 132. The external processor 132 can be configured to process the data transmitted by the injection device 102. The external device 130 can be configured to display (e.g., through a graphical user interface) the data received from the injection device 102 and processed by the external processor 132.

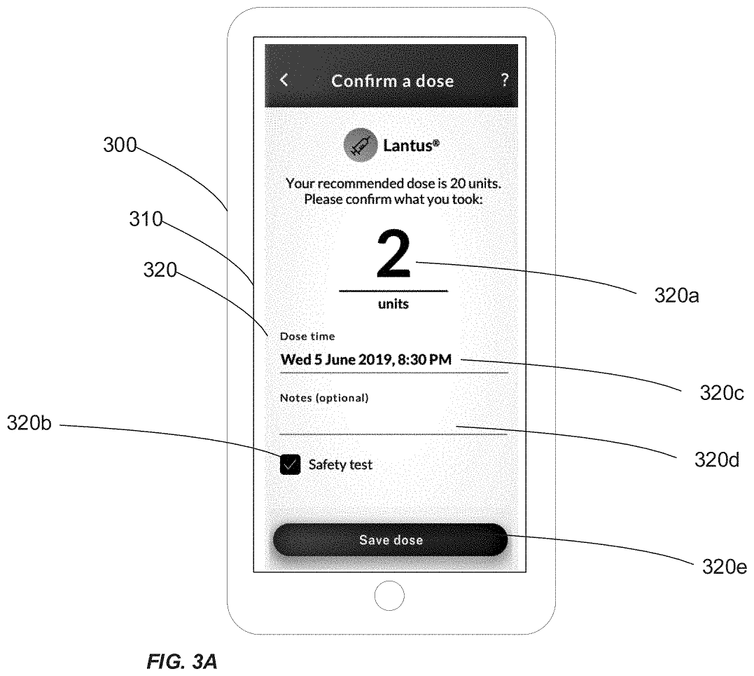

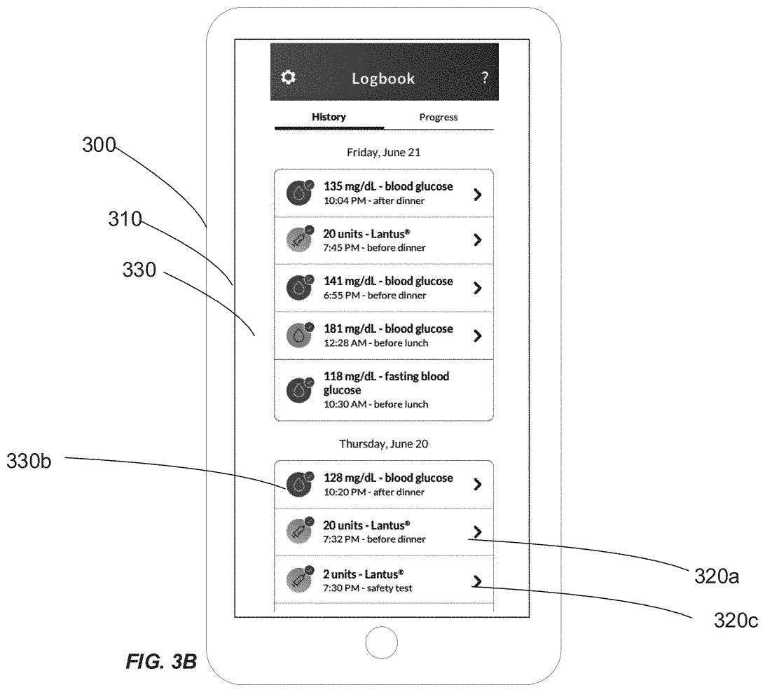

In some implementations, the processor 132 is configured to execute computer-readable instructions to perform one or more operations. In some implementations, the one or more operations include receiving dosing information from the injection device 102 that indicates an amount of medicament dialed or dispensed by the injection device 102. In some implementations, the one or more operations include automatically selecting, based on the amount of medicament, a dose type of a plurality of potential dose types. In some implementations, the plurality of potential dose types includes a priming dose and an injection dose. In some implementations, the one or more operations include rendering a user interface on a display of the external device 130 indicating the selected dose type. In some implementations, the user interface comprises one or more user selectable icons that allows a user to confirm the selected dose type. In some implementations, the user interface comprises one or more user selectable icons that allow a user to adjust the dose type from the selected dose type. In some implementations, the external device 130 can track dosage information received over time, and combine the dosing information with other types of information, such as blood glucose measurement data received from glucometers. In such implementations, the external device 130 can display a user interface showing the medicament amounts over time combined with glucometer readings. Example of user interfaces that can be rendered by the external device 130 are described later with reference to FIGS. 3A-3B.

In some implementations, selecting a dose type includes determining if the amount of medicament exceeds a threshold amount. Selecting a dose type includes selecting a priming dose as the dose type if the amount of medicament does not exceed the threshold amount. For example, in many uses of insulin injection devices, a recommended priming dose can include 2-4 IUs of medicament. Accordingly, if the amount of medicament indicated by the received dosing information is less than 4 IUs, a priming dose can be selected as the dose type. The threshold can be selected based on the type of medicament, error tolerances, a particular prescription, and so forth. For example, for a particular medicament in which corresponding injection doses tend to be relatively small (such as bolus insulin), the threshold dosing amount can be 1 IU or less. In some cases, for such medicaments with relatively small corresponding injection doses, the external device 130 is configured to always select an injection dose as the type of dose. As another example, if the prescribed injection dose is relatively small (e.g., 4 IUs), the threshold dosing amount can be 1 IU or less, or the external device 130 can be configured to always select an injection dose as the type of dose under such circumstances.

As shown in FIG. 1, the needle assembly 115 includes a needle 113 capable of being affixed to the needle hub of housing 110. The needle 113 can be covered by an inner needle cap 116 and an outer needle cap 117, which in turn can be covered by a cap 118. Prior to injection, the caps 116, 117, 118 are removed. When the needle 113 is inserted into a skin portion of a patient, and then the injection button 120 is pushed, the medicament dose displayed in the dosage window 114 is ejected from injection device 102. When the needle 113 of injection device 102 remains for a certain time in the skin portion after the injection button 120 is pushed, a high percentage (e.g., at least 90%) of the dose can be injected into the patient's body. Ejection of the medicament dose can generate a mechanical click sound, which can be different from the sounds produced when using the dosage knob 112.

In some implementations, the electronic components of the electronics assembly 105 are integrated within the housing 110 at a single location, or at multiple locations (e.g., within or attached to a plunger rod 108, and a cavity in the plunger head 109). In some implementations, one or more components of the electronics assembly 105 are included within the stopper 107. In some implementations, one or more components of the electronics assembly 105 are included within the plunger head 109.

In some implementations, at least one of the location of the energy source 104, or the location of one or more electronic components of the electronics assembly 105, are selected independent from the coupling between the electronics assembly 105 and the energy source 104. In some implementations, one or more characteristics of one or more electronic components of the electronics assembly 105, or one or more characteristics of the energy source 104, are selected to couple or uncouple the electronics assembly 105 from the energy source 104.

In some implementations, the housing 110 of the injection device 102 is configured to be separated or broken in multiple segments to provide a user access to the energy source 104, to enable separate disposal of the energy source 104. In some implementations, the medicament reservoir 106 to be assembled with the injection device 102 is manufactured with an inserted stopper 107, is filled with the fluid medicament, and is closed with a crimp seal.

FIG. 2 illustrates a stopper 207 having an electronics assembly 210, according to an embodiment of the present disclosure. In some implementations, the stopper 107 discussed previously with reference to FIG. 1 is substantially similar to the stopper 207 shown in FIG. 2. The stopper 207 includes an expandable rubber material (e.g., neoprene, M18, silicone rubber, etc.). The stopper 207 includes an electronics assembly 210. In some implementations, the electronics assembly 105 discussed previously with reference to FIG. 1 is substantially similar to the electronics assembly 210 shown in FIG. 2.

The electronics assembly 210 includes a pressure or force sensor 215, a position sensor 216, a processor 213, a memory 214, a wireless module 211, and a power module 212. The pressure or force sensor 215 and position sensor 216 are arranged in the electronics assembly 210 such that, when the electronics assembly 210 is disposed in the stopper 207, the position sensor 216 is able to send and receive a sensing signal into the inner volume of the medicament reservoir 106 or otherwise detect the position of the stopper 207 or the plunger rod 108 (or plunger head 109), and the pressure or force sensor 215 is able to measure the force applied to the stopper 207 (or the electronics assembly 210) via the plunger head 109 of the injection device 102 or otherwise to detect the pressure in the plunger rod 108. The processor 213 is operably coupled to all of the elements of the electronics assembly 210 and controls activation of the pressure sensor 215, the position sensor 216, and the wireless module 211. The memory 214 stores instructions for use by the processor 213 in operating the components of the electronics assembly 210.

While FIG. 2 illustrates the electronics assembly 210 in the stopper 207 with the pressure or force sensor 215 integral to the electronics assembly 210, in other instances, the pressure or force sensor 215 is external to the electronics assembly 210 (e.g., located at the point of contact by the plunger rod 108, attached to the plunger rod 108 itself, etc.). While FIG. 2 shows the electronics assembly 210 inside the stopper 207, in some implementations, the electronics assembly 210, with or without an internal pressure or force sensor 215, is located elsewhere in the injection device 102 such that it is capable of receiving a signal from the pressure or force sensor 215.

The wireless module 211 is configured to communicate with an external electronic device in order to communicate information from the electronics assembly 210. In some implementations, the wireless module 211 includes an antenna (or transceiver), such as the antenna 128c discussed previously with reference to FIG. 1. The power module 212 includes an energy source, such as the energy source 104 discussed previously with reference to FIG. 1, and is configured to provide electric power to all of the components of the electronics assembly 210. The power module 212 also includes an electromechanical switch 610, which can be substantially similar to the electromechanical switch 127 discussed earlier with reference to FIG. 1. The electromechanical switch is configured to activate the power module 212 upon the application of a force on the electromechanical switch 610. For example, when a force is applied to the electromechanical switch 610, the electromechanical switch 610 can become activated to generate and transmit an electric signal, which can be received by the power module 212. When the power module 212 receives the electric signal, it becomes activated. Upon activation, the power module 212 provides electric power to the components of the electronics assembly 210. In some implementations, the electronics assembly 210 includes a capacitive device, which includes capacitive circuitry configured to receive power wirelessly from, for example, a smartphone via a nearfield communication protocol (NFC) signal, or by a typical wireless charging device with other means of inductive loading, in order to provide energy to the power module 210.

While FIG. 2 illustrates the electronics assembly 210 including a wireless module 211, in some instances there is no wireless connectivity, and injection device 102 or the electronics assembly 210 itself contains an alert mechanism configured to visually or audibly alert a user or provide certain information to a user. For example, a representative alert mechanism could be a display or series or LED lights arranged to illuminate a different color to a user based on the sensed pressure or force signal and a determined indication of the quality of a drug delivery operation. For instances, a blue light could indicate that the device is ready to use, an orange light could be illuminated while the drug delivery device is conducting the drug delivery operation, and then a green or red light could come on at the sensed completion of the drug delivery operation to indicate a successful or failed delivery operation, respectively. In other instances, the alert mechanism could produce different sounds or beeps to convey the same or different information as the visual alert mechanism.

The electronics assembly 210, through the use of the sensors 215,216, can detect an amount of medicament expelled from the injection device 102. For example, through the use of the position sensor 216, the electronics assembly 210 can detect the location of the stopper 207 in the medicament reservoir 106 (or a change of location), and based on this information, determine an amount of medicament expelled. Additionally, or alternatively, the electronics assembly 210 can use the force (or pressure) sensor 215 to determine how much force is being applied to the stopper 207 (or an amount of time the force is being applied to the stopper 207) to determine an amount of medicament expelled.

As briefly described above, in some implementations, the switch 610 can be activated to power the electronics assembly 210 by a user performing a function that the user already performs during the operation of the injection device 102 for dispensing a medicament (e.g., operating the plunger rod 108 or opening a sealed package that includes the injection device 102), such that additional steps (e.g., manually pushing an additional push-button) may not be necessary to power the electronics assembly 210.

FIGS. 3A-3B illustrate user interfaces 320, 330 for tracking dosage information, according to one or more implementations of the present disclosure. Referring to FIG. 3A, an external device 300 is shown. In some implementations, the external device 130 discussed previously with reference to FIG. 1 includes the shown external device 300 of FIG. 3A. The shown external device 300 is a smartphone, however in other implementations, the external device 300 can be other types of computing devices, such as a tablet, laptop computer, desktop computer, and so forth. The external device 300 includes a display 310, which includes a touchscreen display.

The external device 300 is rendering a user interface 320 on the display 310 after receiving dosing information from an injection device, as described previously with reference to FIG. 1. As shown, user interface 320 includes one or more first graphical representations 320a that indicate an amount of medicament dispensed or dialed by the injection device based on the received dosing information. In the shown implementation, the amount of medicament dispensed is 2 IUs. The user interface includes one or more second graphical representations 320b that includes a user-selectable icon and indicating the type of dose the external device 300 selected for the received dosing information based on the amount of medicament dispensed or dialed. As shown, because the dosing information indicates that the amount of medicament dispensed or dialed was 2 IUs, the external device 300 has automatically caused the user-selectable icon to indicate that a priming dose (“safety test”) was dispensed or dialed. If the user determines that the amount of medicament dispensed or dialed was not a priming dose (e.g., it was an injection dose), the user can select (e.g., tap) the user-selectable icon, and the user-selectable icon will remove the indication that the medicament amount was a priming dose.

In some implementations, the system may determine that a dose is a priming dose if the total scheduled dose is greater than a first threshold amount (e.g., greater than or equal to 4 IU) and the expelled dose is less than a second threshold amount (e.g. less than or equal to 2 IU). In some implementations the first and second threshold amounts may be determined, at least in part based on the accuracy of the position sensor 216. (e.g. ±1 or 2 units).

In some implementations, the external device 300 may check a history of previous doses to determine whether the current dose is a priming dose or a split dose (a single prescribed dose that is split over multiple injections). For example, in some implementations, a patient may provide a partial dose at a given time, and then later, add another dose, the later dose may be relatively small (e.g. within the threshold for triggering the dose as being a priming dose). The external device 300 may determine that if an earlier dose happened within a threshold period of time, for example, within the last hour, that the dose is not a priming dose, but is instead part of a split dose. Otherwise, the external device 300 may determine that the dose is a priming dose.

The user interface 320 also includes a first field 320c that indicates timing information corresponding with the received dosing information. The user interface 320 includes a second field 320d that allows the user to manually input (e.g., using the touchscreen) notes that correspond with the received dosing information. When the user is satisfied with the information presented on the user interface, the user can select a third graphical representation 320e, that includes a user-selectable icon, to save the dosing information for tracking.

Referring to FIG. 3B, the external device 300 is rendering a user interface 330 that integrates the dosing information received over (and saved by the user) with other types of information received from other devices, such as a glucometer. In the shown implementation, the user interface 330 includes a first graphical representation 320a indicating a medicament amount dispensed or dialed and date/time information. The user interface 330 includes a second graphical representation 320b indicating glucometer information. The user interface 330 includes a third graphical representation 320c indicating a medicament amount dispensed or dialed, date/time information, and that the dose corresponding with that medicament amount was determined to be a priming dose.

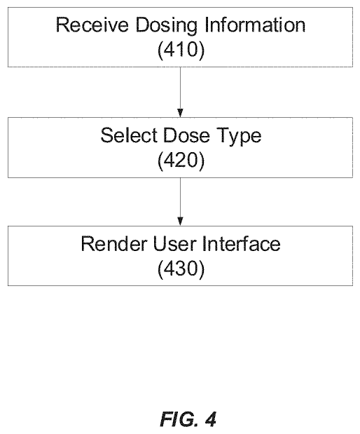

FIG. 4 illustrates a method 400 for tracking dosage information, according to one or more implementations of the present disclosure. In some implementations, the method 400 is performed by one or more of the external devices described in this specification, such as the external device 130 described previously with reference to FIG. 1 and the external device 300 described previously with reference to FIG. 3. The method 400 includes receiving dosing information (block 410), selecting a dose type (block 420), and rendering a user interface (block 430).

At block 410, dosing information is received from an injection device, such as the injection device 102 described previously with reference to FIG. 1. The dosing information can indicate an amount of medicament dispensed or dialed by the injection device.

At block 420, a dose type is automatically selected from a plurality of dose types. In some implementations, the plurality of dose types includes a priming dose (“safety test”) or an injection dose. In some implementations, this selection includes determining if the amount of medicament dispensed or dialed exceeds a threshold amount. In some implementations, if the amount of medicament dispensed or dialed does not exceed the threshold amount, a priming dose is selected. For example, the threshold amount can be 3 IUs, and if the amount of dialed or dispensed medicament is less than 3 IUs, a priming dose is selected as the dose type.

The threshold can be selected based on the type of medicament, error tolerances, a particular prescription, and so forth. For example, for a particular medicament in which corresponding injection doses tend to be relatively small (such as a bolus insulin dose), the threshold dosing amount can be 1 IU or less. In some cases, for such medicaments with relatively small corresponding injection doses, the external device 130 is configured to always select an injection dose as the type of dose. As another example, if the prescribed injection dose is relatively small (e.g., 4 IUs), the threshold dosing amount can be 1 IU or less, or the external device 130 can be configured to always select an injection dose as the type of dose under such circumstances.

At block 430, a user interface is rendered on a display. The user interface indicates the selected dose type. The user interface can also indicate the amount of medicament dispensed or dialed. In some implementations, the user interface includes one or more user selectable icons that allow a user to confirm the selected dose type (see FIG. 3A). In some implementations, the user interface includes one or more user selectable icons that allow a user to adjust the dose type from the selected dose type (see FIG. 3A).

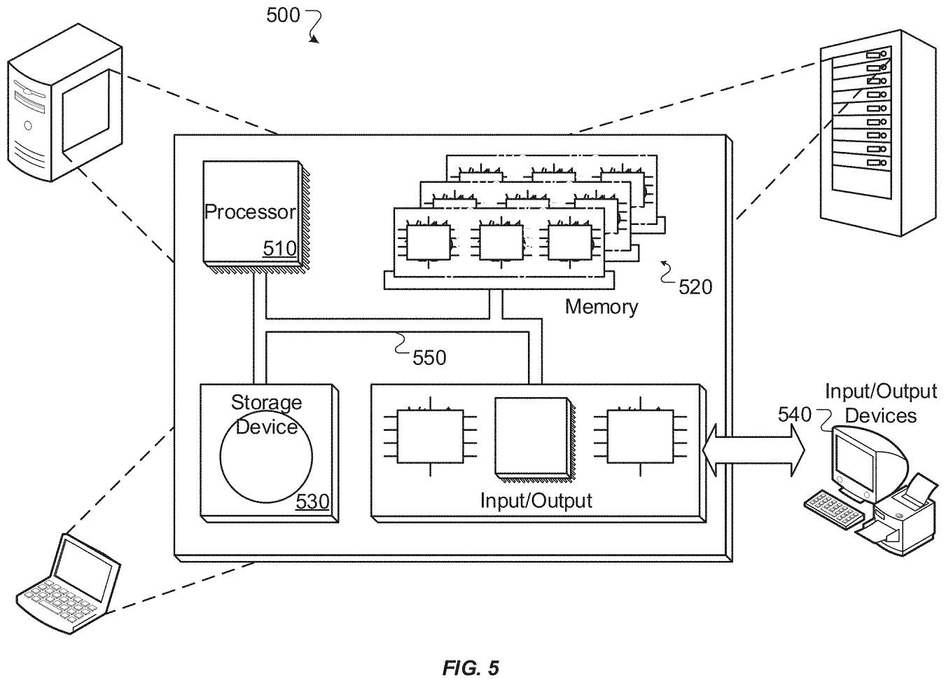

FIG. 5 shows a schematic diagram of an example computing system 500. The system 500 can be used for the operations described in association with the implementations described herein. For example, the system 500 may be included in any or all of the external devices discussed herein. The system 500 includes a processor 510, a memory 520, a storage device 530, and an input/output device 540. Each of the components 510, 520, 530, and 540 are interconnected using a system bus 550. The processor 510 is capable of processing instructions for execution within the system 500. In one implementation, the processor 510 is a single-threaded processor. In another implementation, the processor 510 is a multi-threaded processor. The processor 510 is capable of processing instructions stored in the memory 520 or on the storage device 530 to display graphical information for a user interface on the input/output device 540.

The memory 520 stores information within the system 500. In one implementation, the memory 520 is a computer-readable medium. In one implementation, the memory 520 is a volatile memory unit. In another implementation, the memory 520 is a non-volatile memory unit. The storage device 530 is capable of providing mass storage for the system 500. In one implementation, the storage device 530 is a computer-readable medium. In various different implementations, the storage device 530 may be a floppy disk device, a hard disk device, an optical disk device, or a tape device. The input/output device 540 provides input/output operations for the system 500. In one implementation, the input/output device 540 includes a keyboard and/or pointing device. In another implementation, the input/output device 540 includes a display unit for displaying graphical user interfaces that enable a user to access data related to an item that is collected, stored and queried as described with reference to FIGS. 1-4B.

The features described can be implemented in digital electronic circuitry, or in computer hardware, firmware, software, or in combinations of them. The apparatus can be implemented in a computer program product tangibly embodied in an information carrier, e.g., in a machine-readable storage device, for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include, e.g., a LAN, a WAN, and the computers and networks forming the Internet.

The computer system can include clients and servers. A client and server are generally remote from each other and typically interact through a network, such as the described one. The relationship of client and server arises by virtue of computer programs running on the respective computers and having a client-server relationship to each other.

In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other implementations are within the scope of the following claims.

The terms priming dose or safety test are used herein to describe a dose corresponding to a priming operation. The term injection dose is used herein to describe a dose corresponding with an injection of liquid into a human or animal. For example, a priming operation can include dialing two IUs of liquid, pointing the injection device upwards, and dispensing the dialed dose of liquid into the surrounding environment (e.g., outside of the human or animal body) such as to remove air pockets within the injection device. An example injection can include dialing twenty IUs of liquid, penetrating the skin of the human or animal with the needle of the injection device, and dispensing the dialed twenty IUs of liquid into the body of the human or animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described in this specification, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short- or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic sub stituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom.

Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen.

Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

A number of implementations of the present disclosure have been described. Nevertheless, it will be understood that various modifications may be made without departing from the spirit and scope of the present disclosure. Accordingly, other implementations are within the scope of the following claims.

What is claimed is:

1. A computer-implemented method of tracking dosing information of an electronically enabled injection device, comprising:
receiving, by a computing device, dosing information indicating an amount of medicament dialed or dispensed by the electronically enabled injection device;
automatically selecting, by the computing device and based on the amount of medicament, a dose type of a plurality of potential dose types; and
rendering, by the computing device and on a display of the computing device, a user interface indicating the selected dose type.

2. The computer-implemented method of claim 1, wherein the plurality of dose types includes a priming dose and an injection dose.

3. The computer-implemented method of claim 1, wherein the user interface comprises one or more user selectable icons that allows a user to confirm the selected dose type.

4. The computer-implemented method of claim 1, wherein the user interface comprises one or more user selectable icons that allow a user to adjust the dose type from the selected dose type.

5. The computer-implemented method of claim 1, wherein:
the plurality of dose types comprises a priming dose and an injection dose; and
selecting a dose type comprises determining if the amount of medicament exceeds a threshold amount.

6. The computer-implemented method of claim 5, wherein selecting a dose type comprises selecting a priming dose as the dose type if the amount of medicament does not exceed the threshold amount.

7. A system for tracking dosing information of an electronically enabled injection device, the system comprising:
one or more processors;
computer memory storing instructions that, when executed by the processors, cause the processors to perform operations comprising:
receiving, by a computing device, dosing information indicating an amount of medicament dialed or dispensed by the electronically enabled injection device;
automatically selecting, by the computing device and based on the amount of medicament, a dose type of a plurality of potential dose types; and
rendering, by the computing device and on a display of the computing device, a user interface indicating the selected dose type.

8. The system of claim 7, wherein the plurality of dose types includes a priming dose and an injection dose.

9. The system of claim 7, wherein the user interface comprises one or more user selectable icons that allows a user to confirm the selected dose type.

10. The system of claim 7, wherein the user interface comprises one or more user selectable icons that allow a user to adjust the dose type from the selected dose type.

11. The system of claim 7, wherein:
the plurality of dose types comprises a priming dose and an injection dose; and
selecting a dose type comprises determining if the amount of medicament exceeds a threshold amount.

12. The system of claim 11, wherein selecting a dose type comprises selecting a priming dose as the dose type if the amount of medicament does not exceed the threshold amount.

13. A computer-readable medium for tracking dosing information of an electronically enabled injection device, the medium storing instructions that, when executed by one or more processors, cause the processors to perform operations comprising:
receiving, by a computing device, dosing information indicating an amount of medicament dialed or dispensed by the electronically enabled injection device;
automatically selecting, by the computing device and based on the amount of medicament, a dose type of a plurality of potential dose types; and
rendering, by the computing device and on a display of the computing device, a user interface indicating the selected dose type.

14. The medium of claim 13, wherein the plurality of dose types includes a priming dose and an injection dose.

15. The medium of claim 13, wherein the user interface comprises one or more user selectable icons that allows a user to confirm the selected dose type.

16. The medium of claim 13, wherein the user interface comprises one or more user selectable icons that allow a user to adjust the dose type from the selected dose type.

17. The medium of claim 13, wherein:
the plurality of dose types comprises a priming dose and an injection dose; and selecting a dose type comprises determining if the amount of medicament exceeds a threshold amount.

18. The medium of claim 17, wherein selecting a dose type comprises selecting a priming dose as the dose type if the amount of medicament does not exceed the threshold amount.
